(19) United States
(12) Patent Application Publication
(10) Pub. No.: US 2008/0234304 A1
(43) Pub. Date: Sep. 25, 2008

(54) N3-Pyridyl-Thiamine And Its Use In Cancer Treatments

(75) Inventors: Jeno Gyuris, Lincoln, MA (US); Ronan C. O'Hagan, Arlington, MA (US); May Han, Brookline, MA (US); Murray Robinson, Boston, MA (US); Solly Weiler, Newton, MA (US)

Correspondence Address: ROPES & GRAY LLP, PATENT DOCKETING 39/361, 1211 AVENUE OF THE AMERICAS, NEW YORK, NY, 10036-8704, US

(73) Assignee: AVEO PHARMACEUTICALS, INC., Cambridge, MA (US)

(21) Appl. No.: 10/594,191

(22) PCT Filed: Mar. 23, 2005
(86) PCT No.: PCT/US2005/009758

§ 371 Date: Apr. 29, 2008

Related U.S. Application Data
(60) Provisional application No. 60/556,219, filed on Mar. 24, 2004.

(51) Int. Cl. A61K 31/51; C12N 5/00; A61P 43/00

(52) U.S. Cl. 514/276; 435/375

(57) ABSTRACT

The invention provides methods using N3-pyridyl-thiamine compounds and pharmaceutical compositions comprising N3-pyridyl-thiamine, which are especially useful for preventing or reducing tumor growth in vivo. The invention is also directed to the benefits of reducing thiamine concentrations, e.g., by means of a thiamine reduced diet, as an effective step in a therapeutic regime for patients treated with N3-pyridyl-thiamine.

### CROSS-REFERENCE TO RELATED APPLICATION

This application claims priority to U.S. Provisional Application Ser. No. 60/556,219 filed Mar. 24, 2004, which is incorporated herein by reference in its entirety.

### FIELD OF THE INVENTION

The invention is directed to methods for reducing tumor growth using N3-pyridyl-thiamine compounds and to pharmaceutical compositions comprising N3-pyridyl-thiamine. The invention is also directed to the benefits of reducing thiamine concentrations, e.g., by means of a thiamine reduced diet, as an effective step in a therapeutic regimen for patients treated with N3-pyridyl-thiamine.

### BACKGROUND OF THE INVENTION

Cancer continues to be a worldwide medical health problem. Because of the need to screen large numbers of animals and the associated high cost involved in drug discovery, many anti-tumor drugs have been screened and initially selected based on promising effects on controlling growth and proliferation in human tumor cell lines. Anti-tumor agents, however, can look promising in cell based assays and yet behave quite differently in reducing tumor growth when administered in vivo. Many cancer drugs, for example, target pathways involved in initiation of tumors, whereas patients most often seek treatment at a stage after tumors have formed. It is thus important to develop and use animal models early in drug development to identify compounds and treatments that will effectively reduce tumor growth in the patient once such tumors have formed. Toxicity is also a major problem with chemotherapeutic agents. It is thus highly desirable to find anti-tumor drugs that will have reduced side effects when administered at concentrations that are effective in reducing tumor growth.

### SUMMARY OF THE INVENTION

The invention provides pharmaceutical compositions comprising N3-pyridyl-thiamine (N3PT) and methods using N3PT or pharmaceutical compositions thereof for reducing tumor growth in a patient. The invention also provides methods for inhibiting cell proliferation and tumor cell growth in vitro and in vivo, and stimulating apoptosis in tumor cells by administering N3PT or a pharmaceutical composition comprising N3PT, either alone or in combination with thiamine-restricted diet and/or other therapeutic agents. Methods for inhibiting transketolase activity, reducing cellular ribose-5-phosphate levels and inhibiting nucleic acid synthesis are also provided.

### BRIEF DESCRIPTION OF THE DRAWINGS

FIG. 1: Schematic drawing of N-3 pyridyl thiamine (N3PT).

FIG. 2: N3PT inhibits growth of tumors in vivo. Tumors were induced in mice at day 0 (Example 1) and mice were then treated at day 7 with vehicle alone or with N3PT. Tumor weight (mg) was measured 11 days after treatment (day 18) with vehicle (V) or 200 mg/kg/day N3PT (N). The averages (av) of each group (N=3 vehicle, N=3 N3PT) are shown.

FIG. 3: Bioluminescent imaging of tumors treated with vehicle alone or N3PT. Bioluminescent tumors were induced in mice as in FIG. 2 and photographed (A) prior to treatment (day 7); and (B) 7 days post-treatment with vehicle (right image) or 200 mg/kg/day N3PT (left image).

FIG. 4: Bioluminescent imaging of tumors treated with vehicle alone or N3PT plotted as relative luminescence as a function of time of treatment (days 0-7). Diamonds are vehicle alone; triangles are 200 mg/kg/day N3PT.

FIG. 5: N3PT treatment has no effect on overall animal weight. Mice treated with vehicle alone, or with 200 mg/kg/day N3PT were weighed at day 0, day 6 and day 11 (Example 1). Weight is shown in grams.

FIG. 6: N3PT treatment has no effect on major organ weight. Mice treated with vehicle alone or with 200 mg/kg/day N3PT were sacrificed, major organs dissected and weighed at day 11 post treatment. Weight is shown in milligrams, for brain, heart, kidney, liver, lung and spleen.

FIG. 7: Schematic of a transketolase enzymatic reaction.

FIG. 8: N3PT inhibits transketolase activity in a cell based assay. Shown is % inhibition of transketolase activity as a function of the log of N3PT concentration (micromolar).

FIG. 9: N3PT selectively inhibits transketolase (TK). Competitive inhibition of TK and kGDH by N3PT, expressed as percentage inhibition as a function of treatment with the compound. Shown is % inhibition of kGDH activity as a function of the log of N3PT concentration (micromolar) (triangles). The transketolase inhibition curve from FIG. 8 is overlayed (circle).

FIG. 10: N3PT is a competitive inhibitor of transketolase. Competitive inhibition of TK by N3PT in cells treated with increasing doses of thiamine, expressed as percentage enzymatic activity (the slope of initial linear range) of controls not treated with compounds. The values (y) were plotted as a function of the log concentration of N3PT (micromolar) (x) and fitted to a sigmoidal dose-response curve.

FIG. 11: Whole blood transketolase activity is reduced in animals treated with N3PT. Results are shown as transketolase activity relative to vehicle average (V-av) (%) for whole blood from animals treated with vehicle alone (N=3) or with 200 mg/kg/day N3PT (N=3), av=average activity of three animals in either group.

FIG. 12: Tumor cell transketolase activity is reduced in animals treated with N3PT. Results are shown as transketolase activity relative to vehicle average (%) for tumors from animals treated with vehicle alone (N=3) or with 200 mg/kg/day N3PT (N=3). V=vehicle, N=N3PT treated animals, L=left tumor, R=right tumor, av=average.

FIG. 13: N3PT is a selective inhibitor of transketolase in vivo. Inhibition of transketolase, kGDH and G6PDH by treatment with vehicle or 200 mg.kg.day N3PT is shown as % average of activity when treated with vehicle alone. N3PT exhibits no effect on G6PDH activity.

FIG. 14: N3PT inhibition on transketolase activity is long lasting in a cell based assay. Competitive inhibition of TK by N3PT, expressed as relative mean transketolase activity as a function of the log of N3PT treatment dose (micromolar) at days 2, 3, 5 and 7. Cells are treated with N3PT for two days after plating (day 2) and washed out. TK activities are monitored everyday thereafter (days 3-7).

FIG. 15: N3PT is a long lasting transketolase inhibitor in vivo. The enzymatic activities of transketolase (TK) in blood and spleen after a single dose of N3-pyridyl thiamine (N3PT) in mice (100 mg/kg, i.p.) from 0 hr to 120 hr after dosing relative to the activity at 0 hr.

FIG. 16: A single dose of N3PT has no effect on G6PDH in vivo. The enzymatic activities of glyceraldehyde-6-phosphate dehydrogenase (G6PDH) in blood, spleen and brain after a single dose of N3PT in mice (100 mg/kg, i.p.) shown from 0 hr to 120 hr after treatment. Activity is represented relative to G6PDH activity at 0 hr.

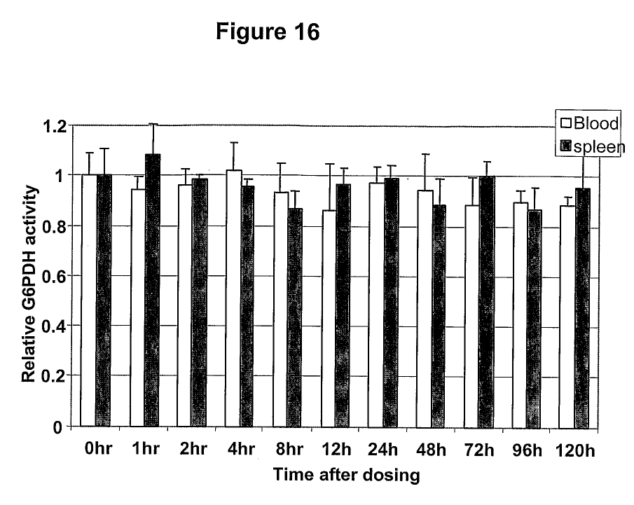

FIG. 17: Low-thiamine diet enhances the sensitivity to N3PT inhibition of TK in spleen. Animals were switched to diets containing 16.5 mg/kg (unchanged), 5 mg/kg, 1 mg/kg, or 0 mg/kg thiamine, from a normal chow containing 16.5 mg/kg thiamine. The recommended daily amount of thiamine for mice is 5 mg/kg. At days 10, 20, and 28 after diet switching, a single or multiple doses of N3PT (bid schedule) were injected ip into the animals. Twelve hours later, tissues were removed and enzymatic activities were recorded. TK activities were normalized to normal diet (containing 16.5 mg/kg thiamine) treated with PBS vehicle.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Worthington Enzyme Manual, Worthington Biochemical Corp. Freehold, N.J.; Handbook of Biochemistry: Section A Proteins, Vol I 1976 CRC Press; Handbook of Biochemistry: Section A Proteins, Vol II 1976 CRC Press; Bast et al., Cancer Medicine, 5th ed., Frei, Emil, editors, BC Decker Inc., Hamilton, Canada (2000); Lodish et al., Molecular Cell Biology, 4th ed., W. H. Freeman & Co., New York (2000); Griffiths et al., Introduction to Genetic Analysis, 7th ed., W. H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000).

The nomenclatures used in connection with, and the laboratory procedures and techniques of cell and tissue culture, molecular biology, cell and cancer biology, virology, immunology, microbiology, genetics, protein and nucleic acid biochemistry, enzymology and medicinal and pharmaceutical chemistry described herein are those well known and commonly used in the art.

All publications, patents and other references mentioned herein are incorporated by reference in their entirety.

The following terms, unless otherwise indicated, shall be understood to have the following meanings:

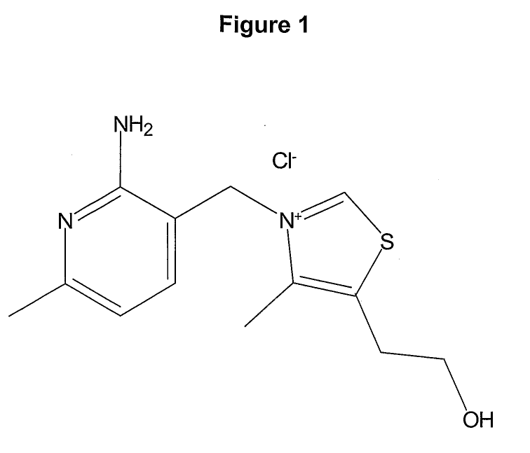

As used herein, the term “N3-pyridyl-thiamine” (or “N3PT”) refers to 3-[(2-amino-6-methyl-3-pyridinyl)methyl]-5-(2-hydroxyethyl)-4-methyl thiazolium. See FIG. 1. The term “N3PT” includes pharmaceutically acceptable salts and derivatives of N3PT.

As used herein, the “activity” of a factor refers to a process or action of excitation or inhibition, and encompasses any specific activity of the factor in question (e.g., specific binding to one or more other cellular factors, and enzymatic activity when referring to enzymes).

As used herein, the term “drug treatment” refers to the general act of administering or applying a drug to a patient for a disease or injury. This act can include but is not limited to the general manipulation and management of factors such as the dosing, concentration and scheduling of the drug regimen so applied. Cancer “therapy” or “treatment” includes any medical intervention resulting in the slowing of tumor growth or reduction in tumor metastases, as well as partial remission of the cancer in order to prolong life expectancy of a patient

As used herein, the term “TPP mimetic” or “TPP mimetic drug” refers to a compound that is similar in both structure and function to a known compound or class of compounds which inhibit thiamine pyrophosphate utilizing enzymes.

As used herein, the term “tumor” refers to a heterogeneous tumor sample in or from a patient that contains a mass of tumorigenic cells and other non-transformed normal cells. “Tumor” may also refer to tumorigenic cells, tumor-derived cells or cell lines derived from any of the above. The term “tumor” encompasses both hard and soft tumors that contain primary or metastatic tumorigenic cells associated with a malignancy. Examples include but are not limited to hematopoietic malignant cells (e.g. lymphomas, leukemias) and other malignant masses derived from specific organs (e.g. fibrosarcomas, carcinomas, hepatomas, and melanomas).

As used herein, the term “tumor-derived cell” refers to a cell extracted from a tumor in an animal that has been cultured separately from the tumor, in vitro or in vivo.

As used herein, the term “patient” refers to a mammal, and preferably, a human.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. In case of conflict, the present specification, including definitions, will control. The materials, methods, and examples are illustrative only and not intended to be limiting.

Throughout this specification and claims, the word “comprise” or variations such as “comprises” or “comprising”, will be understood to imply the inclusion of a stated integer or group of integers but not the exclusion of any other integer or group of integers.

### N3PT Inhibits Tumor Growth in an Animal Xenograft Model

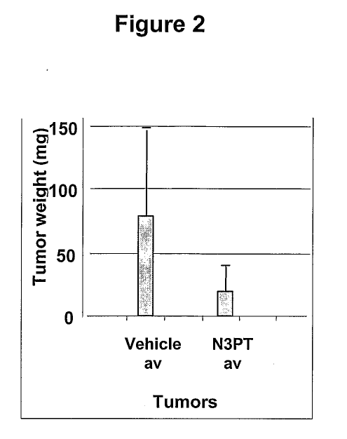

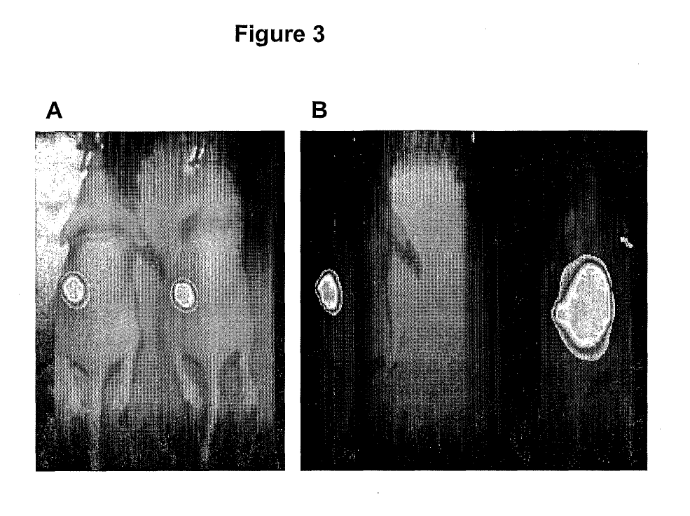

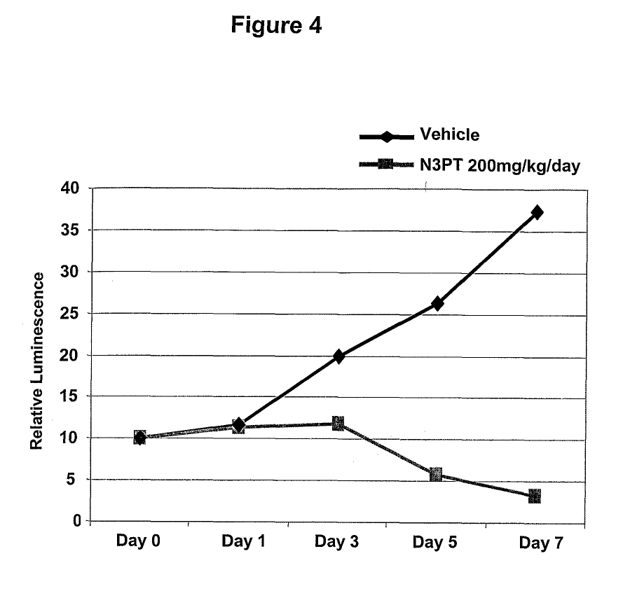

The in vivo efficacy of N3-pyridyl-thiamine (“N3PT”) (FIG. 1) was tested in a mouse xenograft model according to Example 1. Marked tumorigenic cells were injected into a mouse and allowed to grow until palpable tumor masses developed. N3PT was administered and tumor progression measured by bioluminescent imaging and tumor volume and mass measurements. As shown in FIG. 2, N3PT inhibited the growth of explant tumors when administered twice daily at 200 mg/kg/day. FIG. 3 shows a bioluminescent imaging experiment of explant tumors prior to treatment and after 7 days of treatment with N3PT (200 mg/kg/day) or with control vehicle. The results (relative luminescence as a function of treatment time) are quantified in FIG. 4.

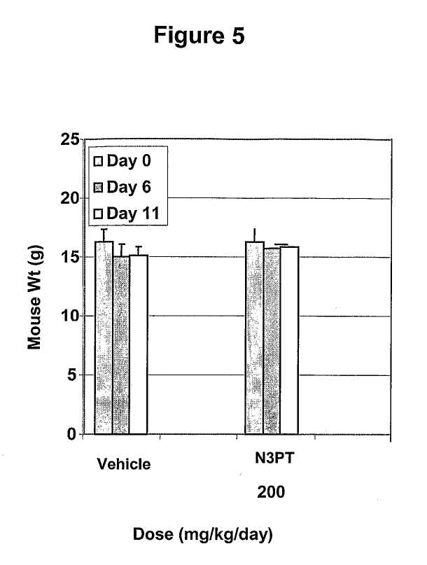

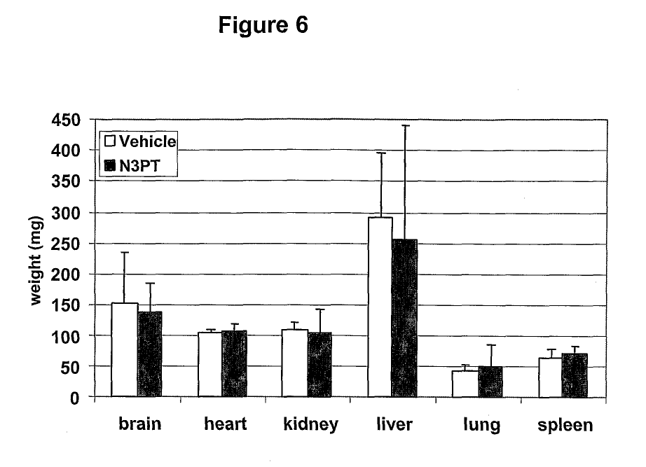

N3PT treatment did not affect overall weight or individual organ weight (as measured in brain, heart, kidney, liver, lung and spleen) in treated animals (see FIGS. 5 and 6), implying a lack of severe toxicity during the treatment. Accordingly, the invention provides N3PT, and pharmaceutical compositions comprising N3PT, as effective therapeutic agents for reducing tumor growth.

Accordingly, the invention provides methods of using N3PT and pharmaceutical compositions comprising N3PT to prevent or treat (i.e., ameliorate, mitigate, alleviate, slow, or inhibit) tumor growth and/or metastasis. The methods may optionally be supplemented with the step of administering at least one additional therapeutic agent, such as a chemotherapeutic agent, antiangiogenic agent or agent which modulates signaling associated with hypoxic conditions in a cell (e.g., fluorouracil, Gemcitabine, Methodtrexate, Cisplatin, Doxorubicin, Toxol, Iritnotecan, Gleevac™, Avastin™ (bevacizumab), angiostatin and endostatin).

### Pharmaceutical Compositions Comprising N3PT

In one embodiment, the invention provides a pharmaceutical composition comprising a therapeutically effective amount of N3PT and a pharmaceutically acceptable carrier. In a preferred embodiment, the composition further comprises at least one additional therapeutic agent such as a chemotherapeutic agent, antiangiogenic agent or agent which modulates signaling associated with hypoxic conditions in a cell. Such pharmaceutical compositions are useful for practicing the methods of the invention, as described in more detail below.

In another preferred embodiment, a therapeutically effective amount of N3PT is coupled to a conjugate that aids in its delivery to a mammal. Preferably, the conjugate aids in delivering N3PT to one or more selective cell types, tissues or organs of the mammal by means of a cellular targeting molecule, e.g., an immunoconjugate or other cell surface specific conjugate. Useful tissues and organs include: lymphatic tissue, blood, brain, kidney, liver, lung, spleen. Useful tissues are not, however, limited to these organs. N3PT is envisioned to be effective for reducing tumor growth in any tumor type in the body.

The conjugate may also aid in controlling the release rate of N3PT. A variety of drug time release agents, compositions and methods are known in the art and may be used by one of skill in the art in conjunction with the pharmaceutical compositions of the invention.

The compositions may comprise or be used in combination with other therapeutic agents, including but not limited to cancer therapeutics such as mitotic inhibitors, alkylating agents, alpha-metabolites, intercalating antibiotics, growth factor inhibitors, cell cycle inhibitors, enzymes, topoisomerase inhibitors, anti-metastatic agents, anti-angiogenic agents and radiation. Exemplary cancer therapeutics are farnesyl transferase inhibitors, tamoxifen, herceptin, taxol, STI571, cisplatin, 5-fluorouracil and cytoxan, some of which specifically target members of the ras tumorigenic pathway. These cancer therapeutics may be administered simultaneously with, prior to, or subsequent to administration of N3PT. These cancer therapeutics may be administered in the same composition comprising N3PT or may be administered in a separate composition.

N3PT may also be used in combination with agents that create a hypoxic environment. Hypoxia, i.e., lack of oxygen, plays a fundamental role in many pathologic processes. In response to hypoxia, cells activate and express multiple genes. Tumor cells may respond to hypoxia by increasingly depending on glycolysis for energy production. The increased rate of glycolysis results in increased concentration of glycolytic metabolites, some of which is channeled into the non-oxidative pentose phosphate pathway by transketolase and converted into ribose-5-phosphate, which is used for nucleic acid synthesis. Some transformed cell lines can also undergo apoptosis in extreme hypoxia and an acidic environment.

Further, without being limited to any particular mechanism of action, the tumor inhibiting effect of N3PT may be associated with inhibition of the non-oxidative pentose phosphate pathway which shuttles carbon from glycolytic reactions to the formation of pentose phosphates used in nucleic acid biosynthesis, including ribulose-5-phosphate and ribose-5-phosphate. Accordingly, in some embodiments, one or more hypoxia-inducing agents are administered simultaneously with, prior to, or subsequent to administration of N3PT. The hypoxia-inducing agent may be administered in the same composition comprising N3PT or may be administered in a separate composition.

As described below, methods of the invention involve administering N3PT or pharmaceutical compositions comprising N3PT to a mammal (e.g., a mouse, a rat, a nonhuman primate, or a human). N3PT is useful in the treatment of hyperproliferative diseases, such as cancers. N3PT is further useful for inhibiting tumor growth, angiogenesis and metastasis. When used to inhibit tumor cell growth, various stages of cancer are treated by these methods, including neoplasia, pre-malignant and malignant tumors. Cancers that can be treated by these methods include, without limitation, cancers that have failed other therapies, cancers at various stages of evolution (including recurring, resistant and minimal residual cancers), cancers whose etiology involves ras, myc, p53, and all other oncogenes whose expression or mis-expression affects signal transduction pathways involved in cell growth, division, proliferation, apoptosis and/or cell death.

More specifically, N3PT is useful for treating lung cancer, bone cancer, pancreatic cancer, skin cancer, cancer of the head and neck, cutaneous or intraocular melanoma, uterine cancer, ovarian cancer, skin cancer, rectal cancer, cancer of the anal region, colorectal cancer, stomach cancer, colon cancer, breast cancer, lung cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), neoplasms of the central nervous system (e.g., primary CNS lymphoma, spinal axis tumors, brain stem gliomas or pituitary adenomas) or glioma, mesothelioma, as well as various leukemias and sarcomas, such as Kaposi's Sarcoma.

A composition of the invention typically contains from about 0.1 to 99% by weight (such as 1 to 20% or 1 to 10%) of N3PT in a pharmaceutically acceptable carrier. Solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch glycolate, and alginic acid. Tablet binders that can be used include acacia, methylcellulose, sodium carboxymethylcellulose, polyvinylpyrrolidone (Povidone™), hydroxypropyl methylcellulose, sucrose, starch, and ethylcellulose. Lubricants that can be used include magnesium stearates, stearic acid, silicone fluid, talc, waxes, oils, and colloidal silica.

Liquid formulations of the compositions for oral administration prepared in water, saline or other aqueous vehicles can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). Physiologically acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically acceptable oil base, such as an ester of a long chain fatty acid (e.g., ethyl oleate).

A topical semi-solid ointment formulation typically contains a concentration of N3PT from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. The optimal percentage of the therapeutic agent in each pharmaceutical formulation varies according to the formulation itself and the therapeutic effect desired in the specific pathologies and correlated therapeutic regimens.

Pharmaceutical formulation is a well-established art, and is further described in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X), the disclosures of which are incorporated herein by reference in their entireties. Conventional methods, known to those of ordinary skill in the art of medicine, can be used to administer the pharmaceutical formulation(s) to the patient.

The pharmaceutical formulation may be administered to the patient by applying to the skin of the patient a transdermal patch containing the pharmaceutical formulation, and leaving the patch in contact with the patient's skin (generally for 1 to 5 hours per patch). Other transdermal routes of administration (e.g., through use of a topically applied cream, ointment, or the like) can be used by applying conventional techniques. The pharmaceutical formulation(s) may also be administered via other conventional routes (e.g., parenteral, subcutaneous, intrapulmonary, transmucosal, intraperitoneal, intrauterine, sublingual, intrathecal, or intramuscular routes) by using standard methods. In addition, the pharmaceutical formulations may be administered to the patient via injectable depot routes of administration such as by using 1-, 3-, or 6-month depot injectable or biodegradable materials and methods.

Regardless of the route of administration, N3PT is typically administered at a daily dosage of from about 1 mg to about 200 mg/kg of body weight of the patient for a few days to achieve a sufficient loading dose, and can be maintained by IV and/or oral dosing. Daily dosing in a human patient will be lower and will generally range from 1 mg to 1 g of i.v. infusion over a 24 hour period. The pharmaceutical formulation can be administered in multiple doses per day, if desired, to achieve the total desired daily dose. The effectiveness of the method of treatment can be assessed by monitoring the patient for known signs or symptoms of the condition being treated (e.g., inhibition of tumor growth) as well as for signs of toxicity.

The pharmaceutical compositions of the invention may be included in a container, package or dispenser alone or as part of a kit with labels and instructions for administration. These compositions can also be used in combination with other cancer therapies involving, e.g., radiation, photosensitizing compounds, anti-neoplastic agents and immunotoxics.

### Methods Involving N3PT Administration

The invention provides methods for reducing tumor growth and/or metastasis by administering to a patient in need thereof N3PT or pharmaceutical compositions comprising N3PT. While not intending to be bound by theory, the following disclosure explores the link between the ability of N3PT to inhibit tumor growth and its ability, both in vitro and in vivo, to inhibit non-oxidative pentose phosphate pathways in cells and tumors. The invention thus provides additional methods for using N3PT and compositions comprising it, based on the biochemical activity of N3PT in a cell.

### N3PT Inhibits Transketolase Activity in Cell Based Assays

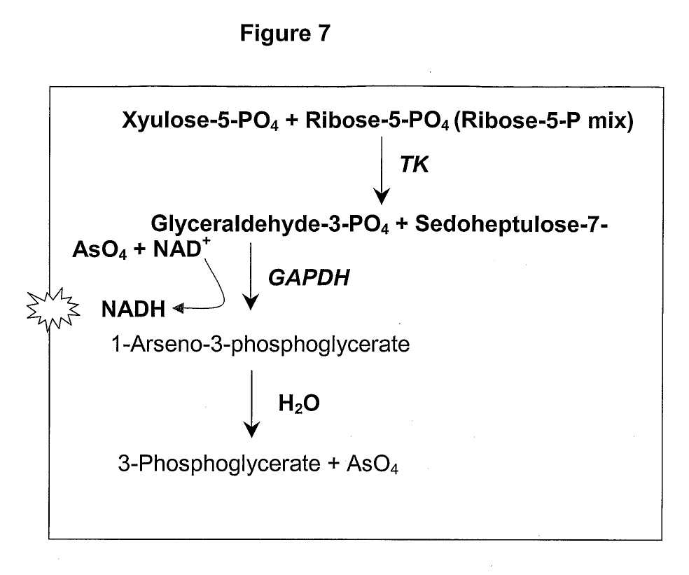

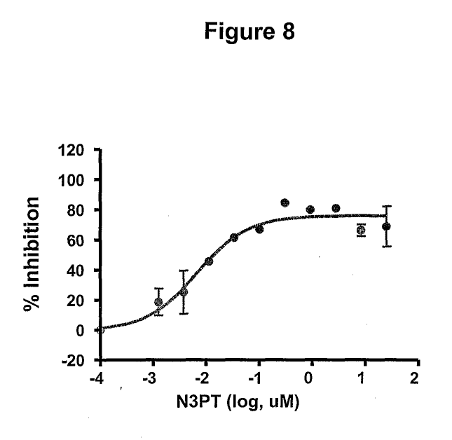

N3PT is a thiamine (vitamin B1) derivative. Thiamine pyrophosphate (TPP) is a co-factor for catalysis by the enzyme transketolase (as well as for the enzymes kGDH and PDH). N3-pyridyl-thiamine (N3PT) (FIG. 1) was identified as being a potent transketolase inhibitor in cell assays (IC₅₀=0.027 μM in cell assays and IC₅₀=0.021 μM with purified apotransketolase) which measure production of glyceraldehyde-3-phosphate by transketolase (FIG. 7). The results of a typical inhibition analysis with N3PT are shown in FIG. 8. A standard method for isolating and measuring the IC₅₀ of apotransketolase is described in Konig et al., J. Biol. Chem. 269: 10879-82 (1994).

### Transketolase Activities Differ in Different Cell Lines

Lysates from a variety of human cancer cells and cell lines were prepared and tested for transketolase enzymatic activity (Example 2; FIG. 7). The following tumor cells and cell lines (all available from the American Type Culture Collection [ATCC] unless otherwise specified) were tested: HCT116 (human colon carcinoma cells having an activated K-ras gene, K-ras^{G13D}); HT1080 (human fibrosarcoma cells); DLD1, Rv22, HCT115, MIA PA CA-2, SK-Mel-5 (from National Cancer Institute [NCI]) and murine cancer lines: R545, LLC, 4T1, and CT26. These experiments showed that transketolase activities differ in different cell lines. Notably, all tumor cells, cell lines and tumors tested expressed high levels of transketolase activity. Normal tissues (blood, brain, heart, kidney, lung and spleen) showed a wide range of activity.

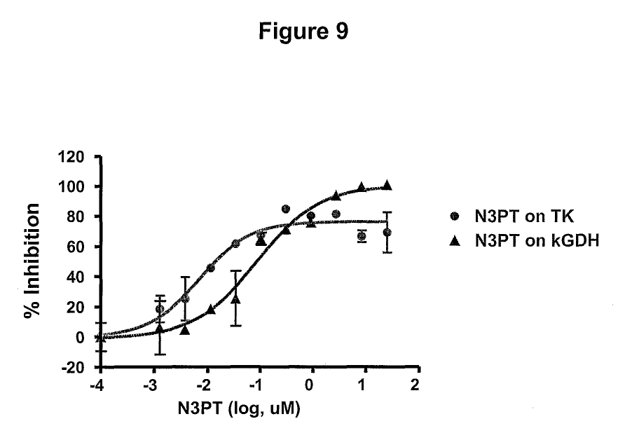

To test the selectivity of N3PT for transketolase, inhibition profiles for transketolase and a different TPP-utilizing enzyme, alpha-ketoglutarate dehydrogenase (kGDH), were compared in extracts from cells incubated with increasing concentrations of N3PT (Example 3). As shown in FIG. 9, N3PT is a more potent inhibitor of transketolase than of kGDH, with the concentrations required to achieve 50% inhibition of enzymatic activity (IC₅₀) differing by an order of magnitude (0.007 for transketolase and 0.086 for kGDH).

### N3PT is a Competitive Inhibitor of Transketolase Activity

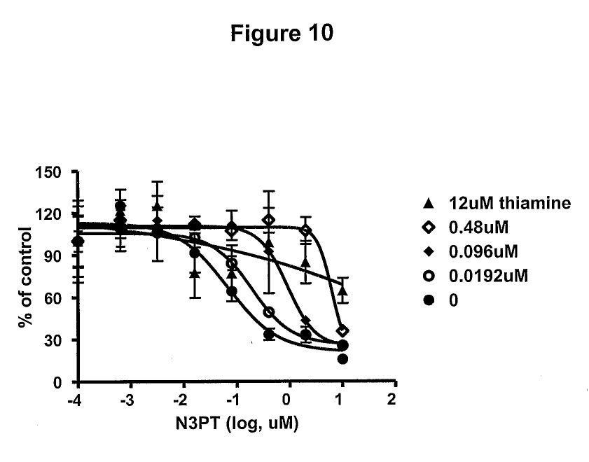

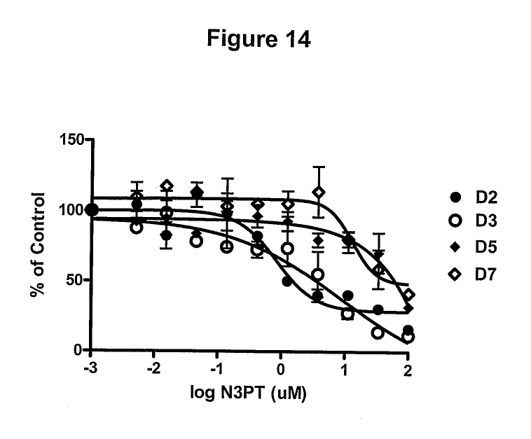

The inhibitory effect of N3PT on transketolase activity was measured in the presence of increasing concentrations of thiamine (Example 3). Inhibition of transketolase by N3PT was reduced by thiamine in a concentration dependent manner, where high levels of thiamine (e.g., 12 μM) were shown to overcome N3PT inhibition (FIG. 10). Inhibition time course experiments indicated that the inhibitory effect of N3PT on cellular transketolase activity persists for several days (FIG. 14).

### N3PT Inhibits Transketolase Activity In Vivo

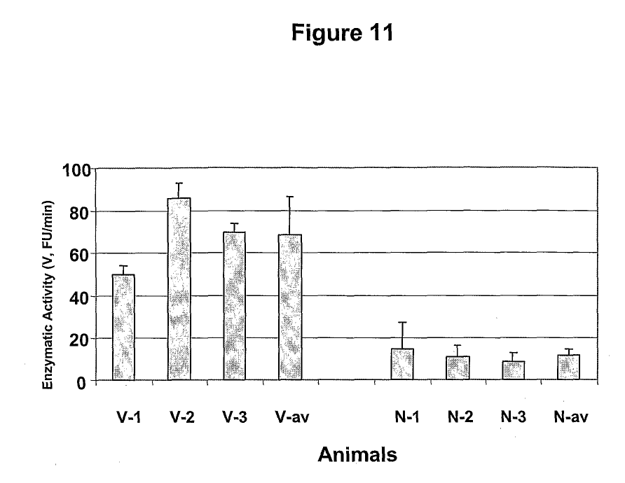

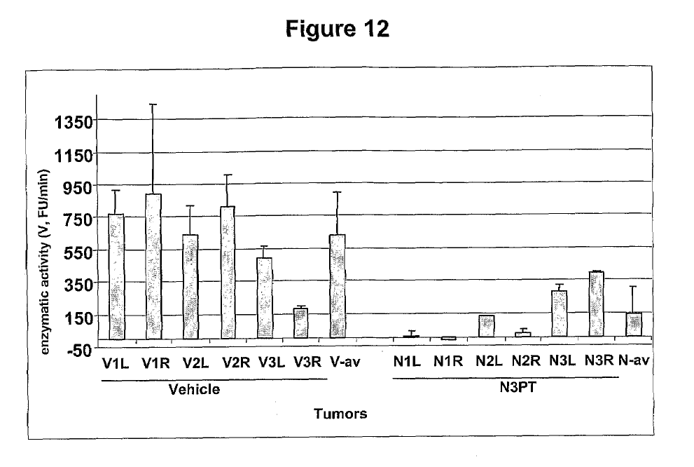

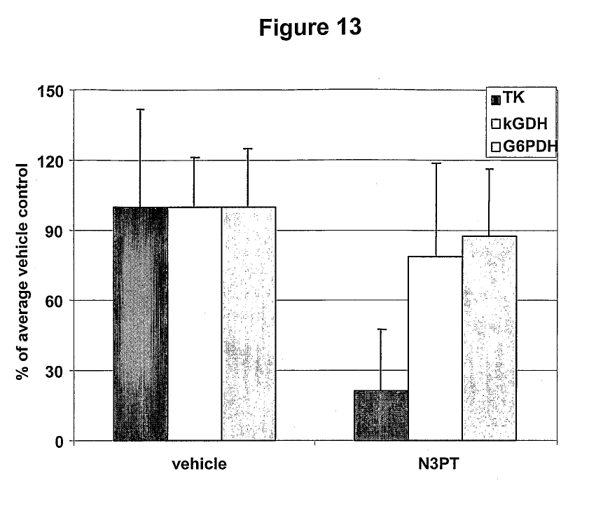

To study the effect of N3PT on transketolase activity in vivo, animals were dosed with a given regimen of N3PT, and blood and various tissue samples isolated and assayed for transketolase enzymatic activity (Example 4). Transketolase activity was measured in whole blood and tumors obtained from N3PT-treated animals (Example 1). As shown in FIG. 11, transketolase activity was selectively inhibited in blood isolated from animals treated with N3PT compared to those treated with control vehicle only. FIG. 12 shows that transketolase activity in tumors is reduced in animals treated with N3PT compared to those treated with control vehicle only. And, as shown in FIG. 13, transketolase activities appear to correlate with tumor weight in the mouse xenograft model at the end of the study (see Example 1).

N3PT administered to a mouse in a single dose (100 mg/kg body weight) selectively inhibits transketolase compared to alpha-ketoglutarate dehydrogenase, another TPP-utilizing enzyme, or glucose-6-phosphate dehydrogenase (G6PDH), for which a single dose of N3PT has little or no effect (FIGS. 13 and 14).

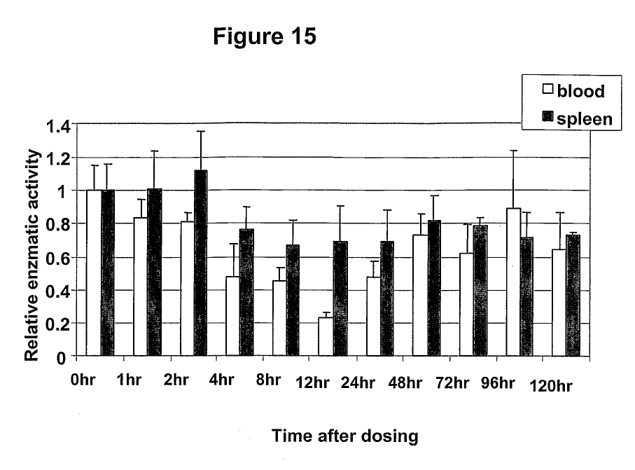

These experiments indicate that N3PT is a potent and long-lasting transketolase inhibitor in vivo which has little or no significant brain penetration after a single dose (FIG. 15). Based on the above, the inhibitory effect of N3PT on transketolase is well correlated with its effect on tumor growth, supporting the notion that inhibition of transketolase directly inhibits tumor growth and maintenance.

The invention thus provides a method for inhibiting transketolase activity in a tumor or tumor-derived cell comprising administering to the cell an effective amount of N3PT. Any amount of detectable inhibition is considered useful as far as achieving a therapeutic effect. In a preferred embodiment, N3PT selectively inhibits transketolase activity compared to its ability to inhibit another TPP-utilizing enzyme (e.g., alpha-ketoglutarate dehydrogenase or pyruvate dehydrogenase).

The amount of N3PT needed to achieve a therapeutic effect will vary depending on the individual tumor and patient treated, and may be determined empirically by one of skill in the art, e.g., by measuring transketolase activity in a tumor biopsy or in the blood of the treated patients. In general, the level will depend on competing levels of thiamine and thiamine-derived compounds such as thiamine pyrophosphate (TPP), which is the cofactor for transketolase (see, e.g., thiamine competition, FIG. 11). The recommended daily allotment (RDA) of thiamine in humans is 1.5 mg. For a human weighing 70 kg, that corresponds to a recommended daily intake of 21 micrograms/kg body weight. The average 20 g mouse which ingests 1 g of food per day (mouse chow, Taklad Global 18%, contains 10 mg thiamine/kg), has a daily intake of about 500 micrograms/kg body weight. The skilled artisan may determine empirically a therapeutically effective range of N3PT by taking into consideration estimated or measured thiamine levels in the mammal to be treated.

Transketolase is known to participate in the non-oxidative pentose phosphate pathway which stimulates ribose biosynthetic pathways and thus increases ribulose-5 phosphate and ribose-5-phosphate production in a cell. In another embodiment, the invention thus provides a method for reducing levels of ribulose-5-phosphate or ribose-5-phosphate in a tumor cell comprising administering to the cell an effective amount of N3PT.

Moreover, the production of pentose phosphates, such as ribulose-5-phosphate and ribose-5-phosphate (a substrate for nucleic acid synthesis), influence nucleic acid biosynthetic rates. Thus, in another embodiment, the invention provides a method for inhibiting nucleic acid synthesis in a tumor cell comprising administering to the cell an effective amount of N3PT.

Increased nucleic acid biosynthesis is required for cell proliferation. Thus in another embodiment, the invention provides a method for inhibiting cell proliferation of a tumor or tumor-derived cell comprising administering to the cell an effective amount of N3PT.

Cancer cells can evolve so that some tumors rely on TK for ribose synthesis to a significantly greater extent than other tumors do. Tumors that rely heavily on TK for ribose synthesis (“TK-reliant tumors”) are more sensitive to treatment with TK inhibitors such as N3PT. Accordingly, some embodiments of the invention include the step of identifying a TK-reliant tumor on or in a mammal, e.g., a human patient, and then administering to the mammal a therapeutically effective amount of a TK inhibitor such as N3PT. TK-reliant tumors are identified by metabolic profiling, which can be performed on tumor biopsy samples or by in vivo metabolic labeling, using conventional techniques. For a review of metabolic profiling techniques, see e.g., Boros et al., 2004, Drug Discovery Today 1:435.

In another embodiment, the invention provides a method for stimulating apoptosis in a tumor or tumor-derived cell comprising administering to the cell an effective amount of N3PT.

Each of the methods of the invention may optionally be supplemented with the step of administering at least one additional chemotherapeutic agent, antiangiogenic agent or agent that induces hypoxic conditions in a cell (e.g. fluorouracil, Gemcitabine, Methodtrexate, Cisplatin, Doxorubicin, Toxol, Iritnotecan, Gleevac™, Avastin™ [bevacizumab], angiostatin, endostatin).

In certain preferred embodiment, each of the above methods is performed on a cell or cells in which the thiamine concentration has been reduced, as described below.

Reducing Thiamine in Conjunction with N3PT Treatment

A typical Western diet is rich in thiamine and many cancer patients take vitamin supplements containing thiamine. As N3PT is a TPP mimetic agent, it will be more effective as an anti-cancer agent when combined with a low-thiamine diet, wherein vitamin supplements that contain thiamine and thiamine-supplemented or thiamine-rich foods are avoided. Any other method for reducing cellular concentrations of thiamine are envisioned to be useful in combination with N3PT treatment methods of the invention.

Accordingly, the invention also provides therapeutic methods which comprise the step of administering N3PT (including a therapeutically effective salt or derivative thereof) or a pharmaceutical composition comprising N3PT to a subject (cell, tissue, organ or mammal) in which the thiamine concentration in the cell or patient has been reduced. Preferably, thiamine concentrations in the subject are limited during the N3PT administration step. More preferably, steps taken to limit thiamine concentrations in the subject are started before the N3PT administration step, e.g., at least 24 hours before, preferably at least 48 hours before, more preferably at least a week before, and most preferably at least two weeks before the N3PT treatment step. In addition, it is preferred that thiamine levels continue to be controlled post N3PT administration, e.g., for at least 24 hours, preferably at least 48 hours, more preferably at least a week, and most preferably at least two weeks after the N3PT treatment step. The recommended minimum thiamine intake level is one that is sufficient to avoid symptoms of toxicity associated with thiamine deficiency. Such symptoms, which are usually mild but can become severe in some instances, include (but are not limited to) those of the cardiovascular and nervous systems such as those associated with wet or dry beriberi or neuropathy and/or Wernicke-Korsakoff syndrome, including peripheral vasodilation, biventricular myocardial failure, sodium and water retention, edema, fulminant cardiovascular collapse, confusion, disordered ocular motility, ataxia of gait, neuropathy and cerebellar degeneration. See, e.g., Singleton and Martin, Current Molecular Medicine 1:197-207 (2001).

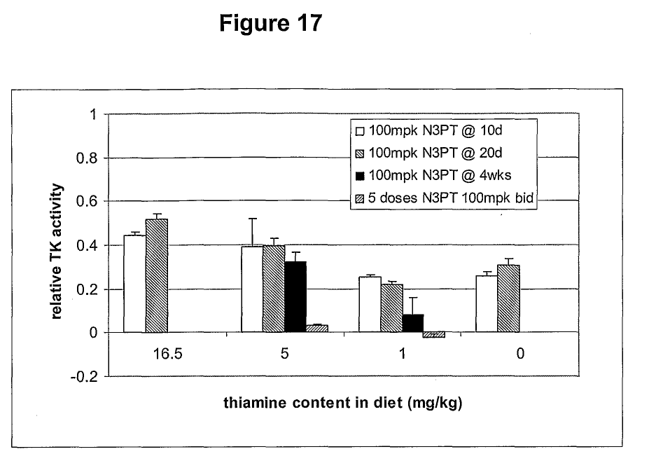

As shown in FIG. 10, decreased amount of thiamine in the culture media greatly enhanced the inhibitory potency of N3PT on TK activity as well as on its anti-proliferative effect (not shown). Animals fed a reduced thiamine diet were also more sensitive to TK inhibition by N3PT (FIG. 17).

The following are examples which illustrate various aspects of the invention. These examples should not be construed as limiting. The examples are included for the purposes of illustration only.

### EXAMPLE 1

### N3PT Inhibition of Tumor Growth In Vivo—The Mouse Xenograft Model

A mouse xenograft model was used to evaluate the efficacy of N3PT in modulating tumor growth. Cancer cells (HCT-116 Luc+, comprising a stably transfected luciferase gene) were injected subcutaneously (10⁶ cells) into Balb C nude mice on both flanks on day 7 prior to treatment. Tumors were allowed to grow to palpable size from test day 0, defined as the start of a treatment regime. N3PT was injected ip (intraperitoneally) at a given regimen (e.g., 200 mg/kg/day) and tumor size monitored by luminance intensity from day 0-7, caliper measurement after day 7, and by weight at the end of study, after the animals were sacrificed. See, e.g., Wang and El-Deiry, Cancer Biol Ther. 2(2):196-202 (2003); Ray et al., Cancer Res. 63(6):1160-5 (2003); see also, e.g., U.S. Pat. Nos. 6,416,960; 6,596,257; and 6,217,847.

### EXAMPLE 2

### Determining Transketolase Activity in Human Carcinoma Cell Lines

Human carcinoma cell lines, such as, but not restricted to, HCT116, HT1080, DLD-1, Rv22, MIA PA CA-2, HCT-15, and murine cell lines, CT26, 4T1 (all cell lines described are available from the ATCC), were cultured in standard conditions (37 C, 5% CO2) in DMEM media (10% FBS, 1% Penicillin-Streptomycin) and harvested at 60-80% confluency by trypsinization. Approximately 2 million cells were collected in an Eppendorf tube and washed 2 times (2×) with PBS by centrifugation and stored at −20° C. as cell pellets. Ice-cold lysis buffer (1 ml) (20 mM HEPES, pH 7.5, 1 mM EDTA, 0.2 g/l Triton X-100 and 0.2 g/L sodium deoxycholate, supplemented with 1 mM DTT and 1 mM PMSF just before use) was added to each cell pellet. Cells were lysed by vortexing and cell extracts assayed for transketolase activity at pH 7.5, by coupling a subsequent glyceraldehyde-3-phosphate dehydrogenase (GAPDH) reaction and measuring conversion of NAD to NADH (FIG. 1).

The assay was carried out as follows. Fifteen ul of 5× assay buffer containing final concentrations of 50 mM HEPES, 40 mM KCl, 2.5 mM MgCl2, 5 mM NaArsenate, 1 mM NAD, 2 unit/ml glyceraldehydes-3-phosphate dehydrogenase (GAPDH) was added to 80 μl of lysate. Reaction kinetics were monitored on a fluorescent plate reader to allow any possible background activity via GAPDH to burn out. Then 5 ul of substrate mix containing final concentrations of 0.5 mM ribose-5-phosphate and 0.5 mM xylulose-5-phosphate was added to initiate the reaction. The reaction kinetics were monitored using a fluorescent plate reader, and the slope of the initial linear range was recorded as the velocity of the reaction (FU/min).

### EXAMPLE 3

### Determining N3PT Inhibition Constants for TK, kGDH and PDH Activities

TK, kGDH and PDH enzymes all utilize thiamine pyrophosphate (TPP) as a co-factor for their catalysis. Conventional cell culture media (e.g., DMEM, RPMI) have high levels of thiamine (12 μM and 3 μM, respectively). High thiamine levels will mask the inhibitory effect of N3PT. Thus, to measure the IC₅₀ of N3PT, a thiamine depleted DMEM, containing all the ingredients of normal DMEM except for thiamine (HyClone, custom order), was used. Thiamine-depleted media (TDM) is made up with thiamine-depleted DMEM, 10% FBS, which contains low amounts of thiamine (estimated to be ˜3-5 nM) and 1% Penicillin-streptomycin.

Log-phase growing cells were trypsinized and resuspended in TDM. Twenty-four hours after seeding, 5 μl of 20× N3PT compound stock solution was added to the cells in 95 μl of TDM. After increasing times of N3PT treatment, cells were either subjected to enzymatic reactions directly or were frozen at −20° C. for future assays. Transketolase reactions were carried out as described in Example 2. kGDH reactions were carried out as follows: Cells were lysed by vortexing and cell extracts assayed for transketolase activity at pH 7.5, by coupling a subsequent glyceraldehyde-3-phosphate dehydrogenase (GAPDH) reaction and measuring conversion of NAD to NADH. TK activities could be measured from ≧5000 cells in most cell lines tested. The kGDH reaction scheme is:

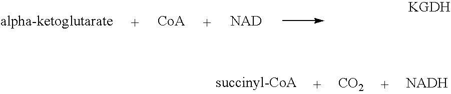

The glucose-6-phosphate dehydrogenase (G6PDH) reaction scheme is:

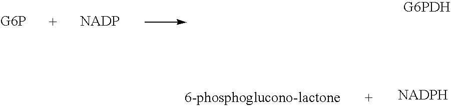

Enzymatic inhibition was expressed as percent of control cells that were not treated with compounds. The values (y) were plotted as function of the log concentration (x) and fitted to a sigmoidal dose-response curve with variable slopes that bears the equation: y=bottom+(top−bottom)/(1+10̂((log EC50−

To assess the extent of competition between N3PT and thiamine, HCT116 cells were plated in thiamine-depleted medium (TDM) 24 hours before treatment with indicated amounts of N3PT and thiamine.

### EXAMPLE 4

### N3PT Inhibition of Transketolase In Vivo

To assess the ability of N3PT to inhibit transketolase in vivo, a single dose of N3PT was administered to Balb C nude mice (ip, 100 mg/kg), and blood and tissue samples were collected over time and enzymatic activities determined. Blood samples were taken (40 μl whole blood) serially or from sacrificed animals at the end of a study. Tissues, including tumor, brain, heart, kidney, liver, lung, spleen, were taken and flash frozen in liquid nitrogen or on dry ice and stored at −80° C. until assay. Blood was diluted into lysis buffer and dissolved. Tissues were suspended in lysis buffer and homogenized with PowerGen 125 (Fisher Scientific) while being submerged in an ice-water bath.

Transketolase assays were performed as in Example 2. The lysate was used right away or flash frozen and stored at −80° C. until assay. The protein concentration in the lysate was determined using a Bradford assay (BioRad) with BSA as a standard. The enzymatic velocity was normalized to the total protein concentration for inter-sample comparisons. In order to obtain reliable results across samples, it was important to have similar starting protein concentrations (such as within 30%). This was achieved by weighing the tissue before lysis so that the suspensions contained similar tissue/ml lysis buffer for all samples. Results are shown in FIGS. 11-15.

### EXAMPLE 5

### Metabolic Profiling

Metabolic profiling using ¹³C-labeled glucose in three in vitro cancer cell lines was carried out using materials and methods generally described in Boros et al., 2004, Drug Discovery Today 1:435. In these experiments it was found that in LAMA84, a chronic myeloid leukemia (CML) cell line, 90% of ribose came from in HCT116 (colon carcinoma) and K562 (another CML line), less than 50% of ribose came from TK.

The ability of N3PT to inhibit proliferation of these cell lines was then tested. LAMA84 was the most sensitive to N3PT, displaying an IC50 of 0.3 μM. The other cell lines, HCT116 and K562, were less sensitive, displaying an IC50 of approximately 2 μM in both cases. LAMA84 also had the highest TK activity (per cell) among these three cancer lines. Good correlations between enzymatic flux and sensitivity to TK inhibitors suggested that it is possible to use metabolic profiling, or any other method that measures enzymatic flux, to determine which cancer patients are most likely to respond well to TK inhibitor treatment.

What is claimed is:

1. A pharmaceutical composition comprising a therapeutically effective amount of N3PT and a pharmaceutically acceptable carrier.

2. The composition of embodiment 1, further comprising at least one chemotherapeutic agent, antiangiogenic agent or agent which modulates signaling associated with hypoxic conditions in a cell.

3. The composition of embodiment 1, wherein the therapeutically effective amount of N3PT is coupled to a conjugate which aids in its delivery to the mammal.

4. The composition of embodiment 3, wherein the conjugate aids in N3PT delivery to a tissue selected from the group consisting of lymphatic tissue, blood, kidney, liver, lung, spleen, brain, prostate, ovary, breast, pancreas, intestine, bladder and skin.

5. A method for inhibiting transketolase activity in a cell comprising administering to the cell an effective amount of N3PT.

6. A method for reducing production of ribulose/ribose-5-phosphate in a cell comprising administering to the cell an effective amount of N3PT.

7. A method for inhibiting nucleic acid synthesis in a cell comprising administering to the cell an effective amount of N3PT.

8. A method for inhibiting cell proliferation comprising administering to the cell an effective amount of N3PT.

9. A method for increasing apoptosis in a tumor cell comprising administering to the cell an effective amount of N3PT.

10. A method for reducing tumor growth in a mammal comprising administering an effective amount of N3PT to the mammal in need thereof.

11. The method of embodiment 10, further comprising administering at least one chemotherapeutic agent, antiangiogenic agent or agent which modulates signaling associated with hypoxic conditions in a cell.

12. The method of embodiment 10 or 11, further comprising the step of limiting thiamine concentrations in the mammal during the administration step.

13. The method of embodiment 12, wherein the mammal is on a reduced thiamine diet during the administration step.

14. The method of embodiment 13, wherein cellular thiamine concentrations are maintained at a level sufficient to avoid toxicity associated with thiamine deficiency.

15. A method for reducing the growth of a tumor in a mammal, comprising the steps of: identifying the tumor as a TK-reliant tumor; and administering to the mammal a therapeutically effective amount of N3PT.

16. The method of claim 15, wherein the step of identifying the tumor as a TK-reliant tumor comprises metabolic profiling.

17. The method of claim 16, further comprising the step of obtaining a tumor biopsy sample.
